(19) United States
(12) Patent Application Publication
(10) Pub. No.: US 2008/0031866 A1
(43) Pub. Date: Feb. 7, 2008

(54) METHOD FOR MODULATING THE PHARMACOKINETICS AND METABOLISM OF A THERAPEUTIC AGENT

(76) Inventor: GARY M. EICHENBAUM, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/764,864

(22) Filed: Jun. 19, 2007

Related U.S. Application Data
(60) Provisional application No. 60/815,223, filed on Jun. 20, 2006.

(51) Int. Cl. A61K 38/43; A61K 38/44; A61K 38/46

(52) U.S. Cl. 424/94.4; 424/94.1; 424/94.6; 424/94.61

(57) ABSTRACT

This invention is a method for enhancing the activity of a therapeutic agent comprising the administration of the agent in combination with an enzyme, whereby a metabolic pathway of the therapeutic agent is counteracted and the agent's pharmacokinetics and metabolism are modulated.

### CROSS-REFERENCE TO RELATED APPLICATIONS

This present application claims benefit of U.S. Provisional Patent Application Ser. No. 60/815,223, filed Jun. 20, 2006, which is incorporated herein by reference in its entirety and for all purposes.

### FIELD OF THE INVENTION

The present invention is directed to a method for enhancing the activity of a therapeutic agent.

In particular, the invention is a method for modulating the pharmacokinetics and metabolism of a therapeutic agent by the administration to a subject in need thereof a therapeutic agent in combination with an enzyme, whereby a metabolite of the agent is transformed back to the agent.

The present invention is further directed to a combination product comprising a therapeutic agent and an enzyme, whereby a metabolite of the agent is transformed back to the agent.

The present invention is also directed to a method for administering to a subject in need thereof a therapeutic agent in combination with an enzyme, whereby a metabolite of the agent is transformed back to the agent.

### BACKGROUND OF THE INVENTION

Compounds that are subject to extensive first pass metabolism and enterohepatic re-circulation can pose significant development and clinical challenges.

Biotransformation of a therapeutic agent to a metabolite of the agent by drug metabolizing enzymes plays an important role in determining the pharmacokinetic profile and/or toxicological effects of many pharmaceutical compounds. Generally, the natural role of metabolizing enzymes is to enable the clearance and in some cases, detoxification, of xenobiotics. However, a drug-metabolizing enzyme can also limit the bioavailability of the therapeutic agent or produce metabolites of the therapeutic agent, which metabolites may cause toxicity. From both a clinical and drug development perspective, extensive metabolism can limit the exposure levels of the therapeutic agent that can be achieved, making it difficult to characterize the dose response and making it more difficult to evaluate toxicity and safety.

The liver was once thought to be the dominant site for the pass metabolism of compounds delivered by the oral route. The intestine also has been shown to play a central role in the metabolism of a number of different drugs. For example, CYP3A4 enzymes in the small intestine have been shown to play an important role in metabolism of drugs such as midazolam, saquanavir and oxybutinin. UDP-glucuronosyltransferase (uridine 5-diphosphate-glucuronosyltransferase) in the intestine has been shown to play an important role in the metabolism of drugs such as the non-steroidal anti-inflammatory drug diclofenac and the immunosuppressive agent mycophenolate mofetil.

Strategies for boosting low bioavailability caused by first pass metabolism have generally fallen into two categories: chemical based and drug delivery based. Chemical based approaches include structural modification of the parent compound, co-administration of an enzyme inhibitor or co-administration of a buffering agent.

Drug delivery based approaches include bypassing duodenal metabolism and delivering a compound to the colon or bypassing the liver through transdermal or parenteral routes.

Although both chemical based and drug delivery based approaches can be effective, they also have inherent limitations.

The limitations of a chemical approach are as follows. Chemically changing the structure of a compound that is in clinical trials can result in a significant setback in terms of time and risk. Chemical modification to eliminate metabolite formation may be impossible in cases where the actual site of metabolism on the molecular structure of the therapeutic agent is essential for the particular activity of a compound. The co-administration of enzyme inhibitors may be of limited value in cases where multiple enzymes form a particular metabolite or where multiple enzymes form multiple metabolites.

For the drug delivery based approach, when there are no regional differences in gut metabolism, it is unlikely that a controlled release delivery would be sufficient to maintain adequate drug levels.

While it has been shown that there are enzymes that catalyze the reverse reaction of a metabolizing enzyme, there remains a need for novel approaches to increase the bioavailability and/or alter the pharmacokinetic profile of a metabolized compound and/or its metabolites by administering an enzyme that transforms or converts a metabolite of a therapeutic agent back to the original agent or a therapeutically available form thereof.

While there a number of endogenous enzymes in the gastrointestinal tract (e.g. CYP450's and beta glucuronidase) oral administration of these enzymes has not been considered. One explanation for this is that the enzyme may be inactivated by the acidic conditions in the stomach, prior to reaching the small intestine.

There have been reports of approaches for reducing the metabolic activity of enzymes in the gastrointestinal track through administration of a compound that inhibits their activity. For example, it has been proposed that reducing the activity of beta-glucuronidase can facilitate the detoxification of carcinogens and tumor promoters/progressors and thereby decrease the risk of carinogenesis (Selkirk J K, Cohen G M and MacLeod M C, glucuronic acid conjugation in the metabolism of chemical carcinogens by rodent cells, Arch. toxicol., 1980, 139:S171-S178 and Walaszek Z, Szemraj J, Narog M, et al. Metabolism, uptake, and excretion of a D-glucaric acid salt and its potential use in cancer prevention. Cancer Detect. Prev., 1997, 21:178-190 as cited in Calcium-D-glucarate (Monograph), Alternative Medicine Review, Aug. 1, 2002).

Accordingly, there still remains a need for increasing the bioavailability and/or altering the pharmacokinetic profile of a metabolized compound and/or its metabolites by oral administration of an enzyme that transforms the metabolite of a therapeutic agent back to the agent.

### SUMMARY OF THE INVENTION

It is an object of the present invention to provide a method for enhancing the activity of a therapeutic agent.

Another object of the present invention is a method for modulating the pharmacokinetics and metabolism of a therapeutic agent by the administration of a therapeutic agent in combination with an enzyme, whereby a metabolite of the agent is transformed back to the agent.

A further object of the present invention is to increase the bioavailability and/or enhance the pharmacokinetic profile of a therapeutic agent and/or its metabolites through the administration of an enzyme, whereby administering the enzyme transforms a metabolite of the agent back to the agent.

A further object of the present invention is to increase the bioavailability and/or enhance the pharmacokinetic profile (e.g. increase the duration of action) of a therapeutic agent and/or its metabolites through the oral administration of an enzyme.

Another object of the present invention is to provide a combination product comprising a therapeutic agent and an enzyme, whereby a metabolite of the agent is transformed back to the agent.

Another object of the present invention is to provide a method for the coadministration of the therapeutic agent with an enzyme, whereby a metabolite of the agent is transformed back to the agent.

Another object of the present invention is to provide a method for administering to a subject in need thereof a therapeutic agent in combination with an enzyme, whereby a metabolite of the agent is transformed back to the agent.

The methods of the present invention increase the bioavailability of a therapeutic agent, which agent is subject to extensive glucuronidation and enterohepatic re-circulation.

Therefore, the bioavailability of a potential cancer therapeutic agent is increased, which agent is subject to extensive glucuronidation and enterohepatic re-circulation, by transforming the metabolite of the agent back to the agent; e.g. by administering a drug metabolizing enzyme such as β-glucuronidase.

### BRIEF DESCRIPTION OF THE FIGURES

FIG. 1 is a schematic diagram illustrating the effect of oral administration of β-glucuronidase on pharmacokinetics and metabolism of a therapeutic agent that undergoes glucuronidation. Section (A) represents duodenal delivery of a therapeutic agent and/or enzyme; Section (B) represents absorption of the agent and intestinal metabolism resulting in formation of a glucuronidated metabolite; Section (C) represents hepatic metabolism resulting in formation of additional glucuronide metabolite; Section (D) represents transport of the agent and glucuronide metabolite into systemic circulation and distribution; Section (E) represents enterohepatic recirculation leading to biliary excretion of the agent and glucuronide metabolite into the duodenum; Section (F) represents cleavage of the glucuronide metabolite to the agent and subsequent reabsorption and return to Section (B); Section (G) represents elimination of the agent and metabolite as feces; Section (H) represents elimination of the agent and metabolite in urine.

### DETAILED DESCRIPTION OF THE INVENTION

The present invention is directed to providing a method for enhancing the activity of a therapeutic agent.

The present invention is directed to a method for modulating the pharmacokinetics and metabolism of a therapeutic agent by the administration to a subject in need thereof a therapeutic agent in combination with an enzyme, whereby a metabolite of the agent is transformed back to the agent.

The present invention is further directed to a combination product comprising a therapeutic agent and an enzyme, whereby a metabolite of the agent is transformed back to the agent.

The present invention is also directed to a method for administering to a subject in need thereof a therapeutic agent in combination with an enzyme, whereby a metabolite of the agent is transformed back to the agent.

The method of the present invention is to increase the bioavailability and/or enhance the pharmacokinetic profile of a metabolized compound and/or its metabolites through the coadministration of an enzyme.

An embodiment of the method of the present invention further comprises the administration of the therapeutic agent in combination with an enzyme for transforming the metabolite of the agent back to the agent, whereby the enzyme releases the agent from a metabolic conjugate form in the small intestine.

An example of the method includes modulating the pharmacokinetics and metabolism of a therapeutic agent by the administration of the therapeutic agent in combination with an enzyme, whereby the therapeutic effectiveness of the agent is boosted.

Another example of the method includes a substantially reduced dose of the therapeutic agent, whereby the therapeutic effectiveness of the agent is below a heretofore known recommended dose and well below toxic levels, thus consequently improving the therapeutic window of said agent.

Another embodiment of the method of the present invention further comprises modulating the pharmacokinetics and metabolism of a therapeutic agent by the administration of the agent in combination with β-glucuronidase, whereby β-glucuronidase releases the agent from a glucuronide conjugate form in the small intestine, thereby modifying the pharmacokinetics and metabolic disposition of the agent.

An advantage of the present invention is that the effective dose of a therapeutic agent can be substantially reduced, since the de-conjugated agent is allowed to enterohepatically recirculate and be reabsorbed into the enterocyte, i.e. the cells of the epithelium, thus subjecting the de-conjugated agent to “second” pass gut and hepatic metabolism.

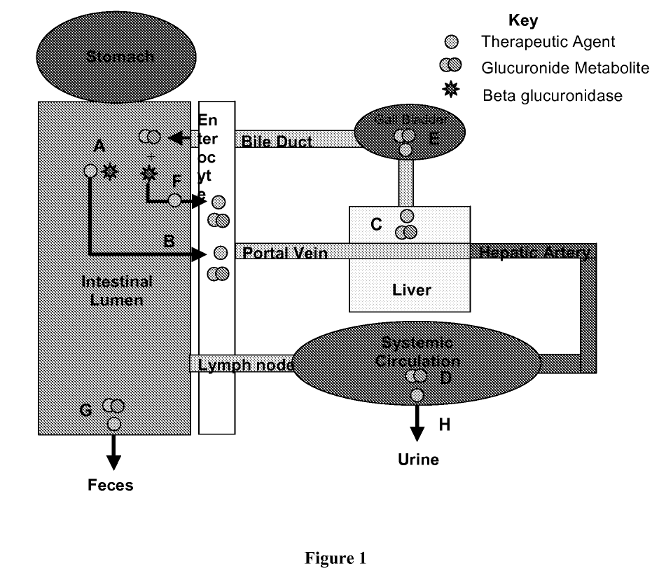

A further embodiment of the method of the present invention further comprises oral coadministration of the enzyme β-glucuronidase and a therapeutic agent, wherein, as shown in FIG. 1, the β-glucuronidase catalyzes hydrolysis of the extensively glucuronidated conjugate agent, thereby increasing the bioavailability of the agent.

A further example of the method includes increasing the bioavailability of a therapeutic agent that typically undergoes glucuronidation in the small intestine and enterohepatic re-circulation, for instance:
an anticholesterol agent including, but not limited to, ZETIA® brand of ezetimibe (Harris M, Davis W, et al., Ezetimibe, Drugs of Today, 2003, 39(4): 229-247);
a cancer therapeutic agent including, but not limited to, irrinotecan, ZARNESTRA® (brand of tipifarnib), histone-deacetylases (HDAC) or (6,7-dimethoxy-2,4-dihydro-indeno [1,2-c]pyrazol-3-yl)-(3-fluoro-phenyl)-amine;
an analgesic agent including, but not limited to, acetominophen, morphine (Fisher M B, Campanale K, et al., In vitro glucuronidation using human liver microsomes and the pore-forming peptide alamethicin, Drug Metab. Dispos., 2000, 28(5): 560-6), codeine (Vree T B, van Dongen R T, et al., Codeine analgesia is due to codeine-6-glucuronide, not morphine, Int. J. Clin. Pract., 2000, 54(6): 395-8) or hydromorphone;
a hormonal agent including, but not limited to, testosterone, dihydrotestosterone, estradiol, 17-alpha-ethynylestradiol or estriol (Fisher, et al.);
an anti-psychotic agent including, but not limited to, risperidone;
a selective estrogen receptor modulator including, but not limited to, tamoxifen or raloxifine;
a retinoid (Fisher, et al.);
micophenolic acid (Papageorgiou C, Enterohepatic recirculation: a powerful incentive for drug discovery in the inosine monophosphate dehydrogenase field, Mini Rev. Med. Chem., 2001, 1(1): 71-7);
a fluoroquinolone including, but not limited to, ciprofloxacin (Tanimura H, Tominaga S, et al., Transfer of ciprofloxacin to bile and determination of biliary metabolites in humans, Arzneimittelforschung, 1986, 36(9): 1417-20; and, Tachibana M, Tanaka M, et al., Acyl glucuronidation of fluoroquinolone antibiotics by the UDP-glucuronosyltransferase 1A subfamily in human liver microsomes, Drug Metab. Dispos., 2005, 33(6): 803-11);
an anticonvulsant agent including, but not limited to, topiramate, carisbamate, valproic acid or GW273293;
an anti-HIV agent including, but not limited to, zidovudine;
a blood glucose lowering agent including, but not limited to, troglitazone or muraglitazar;
an antidiabetic agent including, but not limited to, muraglitzar;
an anti-Parkinson's disease therapeutic agent including, but not limited to, tolcapone; or
an antismoking agent including, but not limited to, nicotine.

A further example of the method includes increasing the bioavailability of a therapeutic agent by administering one of the enzymes selected from the group consisting of N-oxide reductase, sulfoxide reductase, esterase, amidase, glucosidase, β-glucoronidase and sulfatase.

A further example of the method includes increasing the bioavailability of a therapeutic agent by administering a combination product of an esterase enzyme and an ester prodrug.

Method of Use

The present invention is directed to a method for transforming a metabolite of a therapeutic agent back to the agent by the administration of an enzyme.

An embodiment of the method for transforming the metabolite of the agent back to the agent includes oral, intra-duodenal or rectal administration of the enzyme.

Accordingly, by transforming the metabolite of the agent back to the therapeutic agent, the systemic concentration or exposure to the therapeutic agent is increased. Increasing in situ bioavailability by coadministering an enzyme such as beta-glucuronidase will allow the administration of a lower dose of the agent, thus for example, mitigating potential toxicities were a higher dose to be administered. Moreover, agents shown to be therapeutically effective only at doses, which exceed recommendation, at which patients are at risk because serious adverse effects have been demonstrated at such doses, may now be made available at lower, safer doses when coadministered with an enzyme according to an embodiment of the present invention.

The term “administering,” or “administration” refers to the application of a therapeutic agent or to application of an enzyme. The term “coadministering” or “coadministration” refers to the application of an enzyme or in combination with an enzyme.

Such application includes administering an effective amount of said agent with one or more enzymes separately at different times during the course of a therapy or concurrently in a combination form, such as in a combination product.

The term “coadministration” or “coadministering” further includes therapeutically or prophylactically administering an effective amount of a combination product comprising a therapeutic agent and an enzyme, wherein the agent and enzyme may be administered at different times during the course of a therapy or concurrently in a combination form.

An example of the administration of the combination product includes and is not limited to the separate delivery of either the agent or the enzyme.

In a case where the agent is an exogenous or endogenous hormone, the scope of the method for use described herein may only involve the step of delivery of an enzyme formulation or enzyme-inducing agent, whereby the delivery of the enzyme transforms the metabolite of the hormone back to the hormone.

An advantage of the method of the present invention includes greater specificity in modulating the metabolism of a therapeutic agent.

The use of the present invention is not limited to naturally occurring endogenous or exogenous enzymes, it also includes the use of bioengineered enzymes. The scope of the combination product and method for use thereof described herein includes, without limitation, a combination of chemistry, delivery and enzymatic approaches to modulate the levels of therapeutic agent and metabolites.

The variety of delivery modes, in some cases, may involve separate delivery of enzyme system and drug and in the cases of endogenous hormones may only involve the delivery of an enzyme formulation or inducing agent. Such various delivery modes would be expected by one skilled in the art to achieve the aim of the invention and are, thus included without limitation.

The term “enzyme” as used herein, refers to a protein molecule that catalyzes chemical reactions of other substances without it being destroyed or substantially altered upon completion of the reactions. The scope of the term includes, without limitation, endogenous and exogenous enzymes. The term further includes naturally occurring enzymes and bioengineered enzymes or mixtures thereof.

Suitable enzymes for administration include, without limitation, cytochrome P450, glucuronyl transferase, sulfontransferase, carboxyl esterase, β-glucuronidase, soluble epoxide hydrolase, 11-β-hydroxysteroid dehydrogenases, aldehyde oxidase, xanthine oxidase and the like.

Bioengineered enzymes, including bioengineered enzymes that have altered activity compared to the naturally occurring enzymes, such as those generated using molecular biology techniques, may also be suitable and are included in the scope of the invention.

The term “enzyme inducing agent” as used herein, refers to an agent for use in converting an inactive form of an enzyme to one possessing metabolic activity. Such agents include, without limitation, activator ions, cofactors, coenzymes, proenzymes, zymogens (enzyme precursors converted to an active enzyme) and the like.

The term “subject” as used herein, refers to a patient, such as an animal, a mammal or a human, who has been the object of treatment, observation or experiment.

The term “therapeutically effective amount” refers to that amount of a therapeutic agent that elicits the biological or medicinal response in an animal or human, that is being sought by a researcher, veterinarian, medical doctor, or other clinician, which includes alleviation of the symptoms of the disease, disorder or condition being treated.

The term “constitutively effective amount” refers to that amount of an enzyme that is required to maintain a therapeutically effective amount of the agent, whereby the metabolite of the agent is transformed back to the agent.

In embodiments of the present invention, the constitutively effective amount of enzyme includes, and is not limited to, that amount of enzyme that maximizes the extent of in vivo conversion of a metabolite of the therapeutic agent to a “de-metabolized” therapeutic agent.

To maximize the extent of conversion and the rate of such conversion, the stoichiometric ratio of enzyme to agent that may be used may range from a catalytic amount to an excess amount, as estimated by a ratio of an amount of agent converted (mmoles converted/minute/kg) based on enzyme units dosed relative to an amount of agent dosed (mmoles/kg).

The term “enzyme unit” (U) refers to unit for the amount of a particular enzyme. One U is defined as that amount of the enzyme that catalyzes the conversion of 1 micro mole of substrate per minute based on specific conversion conditions, such as at a temperature of 30° C. and a pH and substrate concentration that yields the maximal substrate conversion rate.

An embodiment of the present invention includes, an amount of β-glucuronidase in a range of from about 0.1 to about 10,000 enzyme units dosed relative to an amount of agent dosed; or, a range of from about 1.0 to about 2,000 units; or, a range of from about 10 to about 2,000 units; or, a range of from about 100 to about 2,000 units; or, a range of from about 1.0 to about 1,000 units; or, a range of from about 10 to about 1,000 units; or, a range of from about 100 to about 1,000 units.

In the context of the present invention, the amount of the enzyme administered will allow administration of a therapeutic agent below a toxic level while maintaining the therapeutic window of the agent such that the therapeutically effective amount is maintained throughout the period of absorption. As a result, an agent that may normally have a small therapeutic window can be given at a lower dose while maintaining therapeutic effectiveness. Additionally, the availability of certain therapeutic agents previously discarded due to an inability to administer same at an other than toxic dose may now be administered using the present invention.

The term “therapeutic window” refers to the dose range of an agent or of its concentration in a bodily system that provides safe, effective therapy.

The term “combination product” refers to the use of a combination product comprising a therapeutic agent and an enzyme transforming the metabolite of the therapeutic agent back into the therapeutic agent.

The term “metabolite” refers to the intermediate or products of a metabolic pathway in the body.

The term “enterohepatic recirculation” is the process whereby a substance re-enters the gastrointestinal tract via the bile either in an unchanged form or after having been metabolized, e.g. being conjugated.

Advantageously, the therapeutically effective amount of a therapeutic agent according to the present invention (as in a combination product) may be a reduced amount of the therapeutic agent compared to the effective amount of the agent otherwise recommended for treating a disease, disorder or condition. It is contemplated that the agent may be administered to a subject before, during or after the time the enzyme is administered.

The term “therapeutic agent” refers to an agent or a combination of agents used to treat a disease, disorder or condition and includes, without limitation, facilitating the eradication of, inhibiting the progression of or promoting stasis of a disease, disorder or condition and includes, without limitation, pro-drugs that are cleaved in situ by a co-administered enzyme.

The present invention includes a pharmaceutical composition comprising an admixture of a therapeutic agent and one or more pharmaceutically acceptable excipients for coadministration with an enzyme, wherein the enzyme is used for transforming the metabolite of the therapeutic agent back into the therapeutic agent, as a combination product to a subject in need thereof.

The present invention includes a process for making a pharmaceutical composition of a therapeutic agent and an optional pharmaceutically acceptable carrier for coadministration with an enzyme. The present invention includes a pharmaceutical composition resulting from the process of mixing the agent and an optional pharmaceutically acceptable carrier. Contemplated processes include both conventional and unconventional pharmaceutical techniques.

The present invention includes a product containing (a) a pharmaceutical composition containing a therapeutically effective amount of a therapeutic agent and (b) a pharmaceutical composition containing a constitutively effective amount of an enzyme as a combined preparation for simultaneous, separate or sequential use.

An embodiment of the present invention includes a product containing (a) a pharmaceutical composition containing a therapeutically effective amount of a therapeutic agent that is subject to glucuronidation and (b) a pharmaceutical composition containing a constitutively effective amount of β-glucuronidase as a combined preparation for simultaneous, separate or sequential use.

An embodiment of the present invention includes a product containing (a) a pharmaceutical composition containing a therapeutically effective amount of a therapeutic agent that is subject to glucuronidation selected from the group consisting of an anticholesterol agent, a cancer therapeutic agent, an analgesic agent, a hormonal agent, a selective estrogen receptor modulator, a retinoid, mycophenolic acid, a fluoroquinolone, an anticonvulsant agent, an anti-HIV agent, a blood glucose lowering agent, an antidiabetic agent, an anti-Parkinson's disease therapeutic agent and an antismoking agent and (b) a pharmaceutical composition containing a constitutively effective amount of an enzyme as a combined preparation for simultaneous, separate or sequential use.

An embodiment of the present invention includes an enzyme selected from the group consisting of N-oxide reductase, sulfoxide reductase, esterase, amidase, glucosidase, β-glucoronidase and sulfatase.

Another embodiment of the present invention includes the enzyme β-glucoronidase.

An embodiment of the present invention includes a product containing (a) a pharmaceutical composition containing a therapeutically effective amount of a therapeutic agent that is subject to glucuronidation selected from the group consisting of ezetimibe, irrinotecan, tipifarnib, acetominophen, morphine, codeine, hydromorphone, testosterone, dihydrotestosterone, estradiol, 17-alpha-ethynylestradiol, estriol, tamoxifen, raloxifine, ciprofloxacin, valproic acid, GW273293, zidovudine, troglitazone, muraglitazar, muraglitzar, tolcapone, nicotine and (6,7-dimethoxy-2,4-dihydro-indeno[1,2-c]pyrazol-3-yl)-(3-fluoro-phenyl)-amine and (b) a pharmaceutical composition containing a constitutively effective amount of β-glucuronidase as a combined preparation for simultaneous, separate or sequential use.

An embodiment of the present invention includes a product containing (a) a pharmaceutical composition containing a therapeutically effective amount of (6,7-dimethoxy-2,4-dihydro-indeno[1,2-c]pyrazol-3-yl)-(3-fluoro-phenyl)-amine and (b) a pharmaceutical composition containing a constitutively effective amount of β-glucuronidase as a combined preparation for simultaneous, separate or sequential use.

Said pharmaceutical composition may take a wide variety of forms to effectuate mode of administration, wherein the mode includes, and is not limited to, site specific administration in the gastrointestinal tract (e.g. through a duodenal or colonic catheter) or in an enteric coated capsule or a controlled release delivery system, would be used to bypass the stomach and deliver both the agent and an enzyme. The composition may be in a dosage unit such as a tablet, pill, capsule, powder, granule, sterile solution, suspension or liposomal delivery system for a plurality of administration modes.

Pharmaceutical compositions suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions.

Formulations useful for liposomal delivery for either the enzyme or the therapeutic agent are known to those skilled in the art and include those employed to stabilize the enzymes, making them amenable to oral delivery such as, without limitation, enteric coated or controlled release formulations, conjugation to polymers or peptides, encapsulation in lipid-based carriers and the like.

Alternatively, the pharmaceutical composition may be presented in a form suitable for once-weekly or once-monthly administration at various times using techniques known to those skilled in the art.

The dosage form contains an effective amount of the therapeutic agent necessary to be therapeutically or prophylactically effective as described above. The pharmaceutical composition may contain from about 0.001 mg to about 1000 mg, preferably from about 0.001 to about 500 mg, of the therapeutic agent and may be constituted into any form suitable for the mode of administration selected for the subject in need.

An example of a contemplated effective amount of the therapeutic agent for a pharmaceutical composition containing the therapeutic agent of the present invention may range from about 0.001 mg to about 300 mg/kg of body weight per day. In another example, the range is from about 0.003 to about 100 mg/kg of body weight per day. In another example, the range is from about 0.005 to about 15 mg/kg of body weight per day. The pharmaceutical composition, medicine or medicament containing the therapeutic agent may be administered according to a dosage regimen of from about 1 to about 5 times per day.

An example of a contemplated effective constitutive amount of the enzyme for a pharmaceutical composition containing the enzyme of the present invention may be in a range of from about 0.1 to about 10,000 enzyme units dosed relative to an amount of agent dosed; or, a range of from about 1.0 to about 2,000 units; or, a range of from about 10 to about 2,000 units; or, a range of from about 100 to about 2,000 units; or, a range of from about 1.0 to about 1,000 units; or, a range of from about 10 to about 1,000 units; or, a range of from about 100 to about 1,000 units. The pharmaceutical composition, medicine or medicament containing the enzyme may be administered according to a dosage regimen of from about 1 to about 5 times per day.

The pharmaceutical composition may also contain the above effective amounts of both the therapeutic agent and the enzyme.

For oral administration, the pharmaceutical composition is preferably in the form of a tablet or capsule containing, e.g., 0.01, 0.05, 0.1, 0.5, 1.0, 2.5, 5.0, 10.0, 15.0, 25.0, 50.0, 100, 150, 200, 250, 500, and 1000 milligrams of the therapeutic agent for the symptomatic adjustment of the dosage to the patient to be treated. Optimal dosages will vary depending on factors associated with the particular patient being treated (e.g., age, weight, diet and time of administration), the severity of the condition being treated, the particular compound being used, the mode of administration and the strength of the preparation. The use of either daily administration or post-periodic dosing may be employed.

### EXAMPLES

In the following examples, use of the enzyme β-glucuronidase coadministered with an active therapeutic agent has demonstrated that the metabolized therapeutic agent is released from its glucuronidated conjugate(s) in the small intestine, thereby modifying the disposition of said agent by converting the metabolized agent to the original agent or a therapeutically available form thereof.

### Example 1

A catheterized rat model was used in which the agent alone or agent and β-glucuronidase enzyme was administered intraduodenally or intracolonically. The intraduodenal and intracolonic dosing was intended to prevent the potential for loss of enzymatic activity when orally dosing the β-glucuronidase (e.g. by degradation in the stomach).

Summary of Results

Following intraduodenal administration of the agent together with β-glucuronidase to catheterized rats, there was a 294% increase in the AUC of the agent from 60±18 (ng.hr)/ml (as shown in Table 8) with no enzyme present to an AUC of the agent of 177±55 (ng.hr)/ml with an enzyme dose of 10 mg/kg (as shown in Table 7).

The Cₘₐₓ of the agent without enzyme present was 75±30 ng/ml (as shown in Table 8) and increased to a Cₘₐₓ of 129±41 ng/ml with enzyme present (as shown in Table 7).

Similarly, the primary glucuronide metabolite AUC increased from 1216±371 (ng.hr)/ml with no enzyme present to an AUC of 8940±2144 (ng.hr)/ml with enzyme present.

The resulting pharmacokinetic data suggested that when the agent and its primary glucuronide (i.e. metabolite) were secreted into the duodenum by the bile duct and were exposed to intraduodenally administered β-glucuronidase in the gut lumen, the glucuronide was cleaved back to the agent, thus allowing the agent to be reabsorbed.

### Example 2

Materials and Methods

Compound and Metabolites

The agent used as the test compound (6,7-dimethoxy-2,4-dihydro-indeno[1,2-c]pyrazol-3-yl)-(3-fluoro-phenyl)-amine (hereinafter referred to as Compound 1) and two of its glucuronide metabolites, GluA and GluB, were tested.

In Vitro Metabolism—Incubation of Glu A and Glu B with β-Glucuronidase

β-glucuronidase derived from E. Coli, glucurase at a concentration of 5,000 units/ml and bovine serum albumin were obtained from Sigma (St. Louis, Mo.). Glu A and Glu B were incubated at a concentration of 500 μM with β-glucuronidase (2,500 units) in 100 mM potassium acetate buffer solution in a 0.5 ml filter tube. The incubations were run in triplicate.

To each filter tube, 65 μl of 100 mM potassium acetate buffer, 5 μl of bovine serum albumin, and 100 μl of glucurase were added (100 mM potassium acetate was added to the control wells). The pH was adjusted to either pH 5.0 using 1N potassium hydroxide. 10 μl of 10 mM GluA or GluB were then added and the filter tubes were incubated for 30 minutes, 60 minutes or 18 hours at 37° C. Following incubation 20 μl of acetonitrile was added to the filter tubes and the filter tubes were centrifuged. The resulting filtrate was then analyzed by HPLC. Glu A was found to be relatively unstable in DMSO, especially after freezing and thawing, therefore, Glu A was prepared fresh.

In Vivo Pharmacokinetics

Dosing Schedule and Formulations

A summary of the dosing schedule for the intraduodenal, intracolonic, IV and portal vein rat studies is shown in Table 1. Two separate IV studies were carried out using two separate lots of animals. IV-A was run on the same lot of animals as the oral gavage study and IV-B was run on the same lot of animals as the intraduodenal and intra-colonic studies.

TABLE 1
In Vivo Pharmacokinetic Studies Dosing Schedule

| | Sampling | | Dose | Conc. | Dose Volume |
|---|---|---|---|---|---|
| Route | Site(s) | Vehicle | (mg/kg) | (mg/mL) | (mL/kg) |
| IV | Retro-orbital | HPCD in DI water | 2 | 1 | 2 |
| | sinus | (20/80 wt %) | | | |
| Oral | Jugular and | PEG-400 | 75 | 7.5 | 10 |
| Gavage | Portal Vein | | | | |
| Duodenal | Carotid Artery | Tween 80 in DI | 10 | 10 | 1 |
| | | water (20/80 wt %) | | | |
| Colonic | Carotid Artery | Tween 80 in DI | 10 | 10 | 1 |
| | | water (20/80 wt %) | | | |

For the oral gavage dosing, Compound 1 was prepared in neat PEG-400. For the intraduodenal and intracolonic dosing solutions, Compound 1 was added to neat Tween 80, the mixture was sonicated for thirty minutes at 45° C. until Compound 1 was completely dissolved and then deionized (DI) water was added to form a solution consisting of 20% Tween 80 and 80% DI water by weight.

For the IV dosing solutions (IV), Compound 1 was prepared by addition to DI water containing 20% hydroxypropyl β-cyclodextran by weight.

For the oral enzyme solutions, β-glucuronidase was added to the final formulation at a concentration of 57,600 units/ml.

In Vivo Dosing

For the IV and oral gavage dosing (sampling at jugular and portal vein), young adult male Sprague Dawley rats weighing 250-400 g were purchased from Hilltop Lab Animals Inc. All rat experiments were conducted under approved animal protocols. The rats for the oral gavage dosing were catheterized at Hilltop Lab Animals Inc, had two chronic catheters for sampling: jugular and portal, were housed in cages of Culex™ Automated Blood Sampler (ABS) and blood samples were collected simultaneously via both cannula and were controlled by the Culex™ ABS control software.

For the intraduodenal and intracolonic dosing, male Sprague Dawley rats weighing 300-400 g were purchased from Charles River Labs. All rat experiments were conducted under approved animal protocols. The rats were catheterized at Charles River Labs and had one or more of the following chronic catheters for dosing and sampling: jugular/carotid, duodenal, and colonic. Rats were limited to only one GI catheter and one vascular catheter. For IV dosing, rats were purchased with jugular and carotid catheters for dosing in the jugular catheter and blood sampling through the carotid catheter. For intraduodenal/intracolonic dosing, rats had one of the GI catheters for dosing and a carotid catheter for blood sampling.

The surgeries were done two weeks prior to start of testing. Rats were fed 4 hours post dose administration. All rats were dosed without anesthesia and sacrificed at the end of the study.

Bioanalysis

A quantitative method for determining the amount of (6,7-dimethoxy-2,4-dihydro-indeno[1,2-c]pyrazol-3-yl)-(3-fluoro-phenyl)-amine Compound 1 and its major metabolites, glucuronide A (Cpd 1-GluA) and glucuronide B (Cpd 1-GluB) (see Scheme A) in rat plasma was developed using phenacetin as an internal standard.

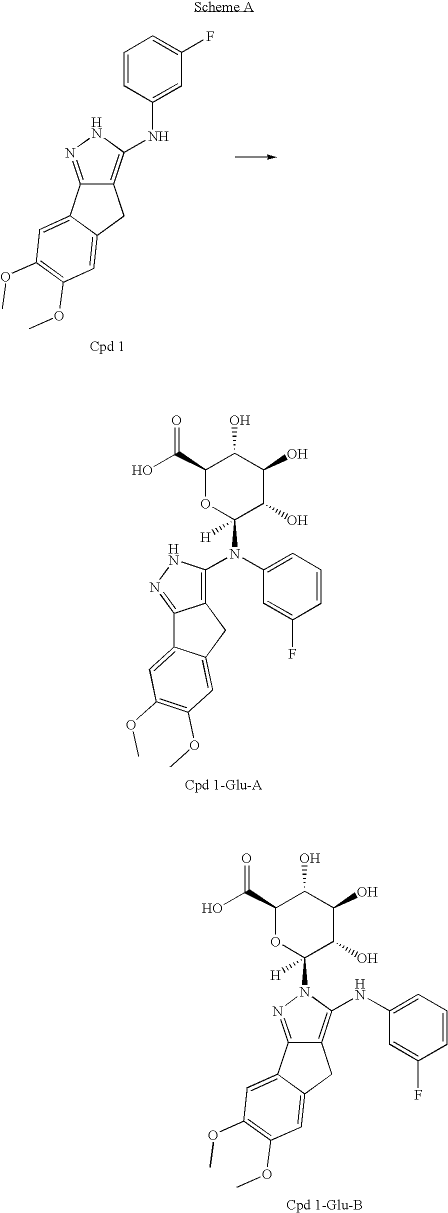

The two glucuronide conjugates were prepared and purified to permit accurate quantification of the levels of intact parent Compound 1 versus Cpd 1-GluA and Cpd 1-GluB. Each standard curve for Compound 1, Cpd 1-GluA, and Cpd 1-GluB generated from rat plasma included ten concentration levels. The lowest limit of quantification for each sample of Compound 1, Cpd 1-GluA, and Cpd 1-GluB was 1.0 ng/ml, with linearity demonstrated to 1000 ng/ml. The quantitative procedure for each sample included: sample preparation, HPLC separation and mass spectrometry detection.

Sample Preparation:

The rat plasma was prepared using protein precipitation techniques. Reference standards and quality control samples in rat plasma for Compound 1 were transferred to a 96 deep-well plate. The precipitation solution contained 100 ng/ml of internal standard in acetonitrile. 300 μl of precipitation solution were added to 100 μl of each standard, quality control and plasma sample. Samples were vortexed for 10 mins, then centrifuged at 4500 RPM for 10 mins. 10 μl of each sample were injected for LC/MS/MS analysis.

HPLC Separation:

The HPLC column chosen for the bioanalysis was purchased from Thermo Electron (Hypersil BetaBasic C18, 5 cm×2.1 mm). The eluting solvents were composed of Solvent A: 0.1% formic acid in H₂O and Solvent B: 0.1% formic acid in acetonitrile. A step gradient was used and flow rate was 300 μl/min. The total run time was 3.2 mins. Solvent B was increased from 35% at 0 min to 75% at 2.1 mins and decreased back to 35% at 2.2 mins. The retention times for Compound 1, phenacetin, Cpd 1-GluA and Cpd 1-GluB were 1.1 min, 0.9 min, 1.3 min, and 1.6 min, respectively.

Mass Spectrometry Detection:

API3000 with a Turbo Ionspray interface from Applied Biosystems was used for the detection of Compound 1, the internal standard and major metabolites. Electrospray ionization was performed in the positive ion mode heated nebulizer with a temperature of 300° C. LC/MS/MS chromatograms were acquired in MRM (multiple reaction mode). The MRM transitions for Compound 1, the internal standard, Cpd 1-GluA and Cpd 1-GluB were from 326.3 to 190.6, 181.1 to 110.3, 502.2 to 368.3 and 502.2 to 326.4, respectively.

Pharmacokinetic Data Analysis

Pharmacokinetic analysis of the plasma concentration data was performed to determine the maximum plasma concentration (Cₘₐₓ) for the oral dose, and the concentration at zero time (C₀; Compound 1 only) by the extrapolation of drug concentrations back to the time of i.v. dosing, the time to maximum plasma concentration (Tₘₐₓ) following the oral dose, area under the plasma concentration vs. time curve (AUC), terminal half-life (t_{1/2}), plasma clearance (CL/F; Compound 1 only), and volume of distribution at steady state (Vdₛₛ; Compound 1 only) for the i.v. dose, using the WinNonlin (Version 3.1, Pharsight, Palo Alto, Calif.) validated computer program.

The oral systemic bioavailability (F) for the oral gavage studies was calculated using Equation (1):

$$F = \frac{{AUC}_{jugular}}{{AUC}_{i . v}} ⨯ \frac{{D}_{i . v}}{{D}_{jugular}} \qquad ( 1 )$$

The intestinal absorption ratio (Fₐ) was calculated by Equation (2):

$${F}_{a} = {Q}_{b} \frac{( AUCjugular - AUCportal )}{Dose} \qquad ( 2 )$$

In Equation (2), Qb is the blood flow and was estimated to be 15.3 mL/min per body weight (250 g) by measurement with a flowmeter.

The hepatic recovery ratio (F_{H}) was calculated using Equation (3):

$${F}_{H} = \frac{F}{{F}_{a}} \qquad ( 3 )$$

Results
Physicochemical Properties

Compound 1 has a MW of 325.35 g/mole and is poorly soluble in aqueous media at pH 7.0 (<0.002 mg/mL) and pH 3.0 (<0.006 mg/mL). A solubility of 7.5 mg/mL was achieved in neat PEG-400 for the oral gavage study. Improved solubility of 10 mg/mL for in vivo dosing studies was achieved by formulating Compound 1 in a solution containing 20/80-weight% Tween 80/DI water and with the exception of the IV dosing. Compound 1 exhibited high absorption (13×10⁻⁶ cm/sec).

I. In Vivo Pharmacokinetics and First Pass Metabolism—No Enzyme Mean Plasma Concentration

Following a single 2 mg/kg IV dose, Compound 1 was rapidly and extensively glucuronidated. The results of samples taken from the retro orbital sinus of male rats (N=4) are shown in Table 2. The Tₘₐₓ for Glu A and Glu B was at 0.3 hours and the Cₘₐₓ for Glu A was 2,970 ng/ml compared with an estimated C₀ for Compound 1 of 783 ng/ml.

TABLE 2
IV Dose (ng/ml)

| Sample | | Cpd 1 | Cpd 1-GluA | Cpd 1-GluB |
|---|---|---|---|---|
| 0 | hrs | 783 | 0 | 0 |
| 0.08 | hrs | 610 | 2443 | 21.2 |
| 0.33 | hrs | 286 | 2970 | 28.4 |
| 1 | hr | 71.3 | 1666 | 17.8 |
| 2 | hrs | 16.3 | 492 | 5.5 |
| 4 | hrs | 1.2 | 68.6 | 0 |
| 8 | hrs | 0 | 369 | 0 |
| 24 | hrs | 0 | 18.6 | 0 |

| | | | |
|---|---|---|---|
| Cₘₐₓ average ± standard | 783 ± 233 | 2,970 ± 322 | 28 ± 3 |
| deviation | | | |
| AUC average ± standard | 345 ± 42 | 8,212 ± 2,397 | 103 ± 47 |
| deviation | | | |
| (ng · hr/ml) | | | |

Following a 75 mg/kg oral gavage dose of Compound 1 and sampling at the jugular (N=4) (Table 3) and portal vein (N=4) (Table 4). The results of samples taken show that Compound 1 was rapidly absorbed and rapidly glucuronidated. The Tₘₐₓ for Compound 1 was at 0.25 hrs and the Tₘₐₓ for Glu A and Glu B was at 0.9 and 0.5 hours, respectively).

The cyclic increase and decrease in the portal vein concentration of Compound 1 between 0.5 and 4 hours are consistent with enterohepatic circulation of the glucuronide conjugates, conversion of the conjugates back to Compound 1, followed by re-absorption across the gut wall.

The values represented in each table for the presence of compound and conjugates are the mean (rounded up to the first decimal place) for a treatment group of 4 animals.

TABLE 3
Oral Gavage Dose Jugular Sample (ng/ml)

| Sample (Jugular) | | Cpd 1 | Cpd 1-GluA | Cpd 1-GluB |
|---|---|---|---|---|
| 0 | hrs | 6.7 | 2.2 | 0 |
| 0.08 | hrs | 447.3 | 2569 | 37.8 |
| 0.25 | hrs | 2115 | 12425 | 168.6 |
| 0.33 | hrs | 1771.3 | 16080 | 233.3 |
| 0.5 | hrs | 1221 | 15710 | 223.1 |
| 0.67 | hrs | 626 | 14660 | 243.5 |
| 1 | hr | 442 | 14340 | 229.9 |
| 1.25 | hrs | 464.6 | 11690 | 178.1 |
| 1.5 | hrs | 409.2 | 11670 | 169.1 |
| 2 | hrs | 377.5 | 11135 | 141 |
| 3 | hrs | 127.4 | 6485 | 90.0 |
| 4 | hrs | 61.3 | 3750 | 51.4 |
| 6 | hrs | 51.1 | 4286.7 | 71.3 |
| 12 | hrs | 22.8 | 5166.7 | 86.5 |
| 24 | hrs | 7.7 | 2473 | 29.0 |

| | | | |
|---|---|---|---|
| Cₘₐₓ average ± standard | 2,115 ± 882 | 17,590 ± 1,937 | 268 ± 52 |
| deviation | | | |
| AUC average ± standard | 2,335 ± 619 | 117,187 ± 21,641 | 1,710 ± 447 |
| deviation | | | |
| (ng · hr/ml) 0 to 24 | | | |
| hours | | | |
| AUC average ± standard | 2,406 ± 601 | 169,279 ± 49,439 | 2,185 ± 640 |
| deviation | | | |
| (ng · hr/ml) 0 to | | | |
| infinite hours | | | |

TABLE 4
Oral Gavage Dose Portal Sample (ng/ml)

| Sample (Portal) | | Cpd 1 | Cpd 1-GluA | Cpd 1-GluB |
|---|---|---|---|---|
| 0 | hrs | 2.4 | 1.3 | 0 |
| 0.08 | hrs | 7525 | 9465 | 82.8 |
| 0.25 | hrs | 4252.7 | 18946.7 | 214.9 |
| 0.33 | hrs | 3880.7 | 22433.3 | 262.7 |
| 0.5 | hrs | 872.9 | 17360 | 191.5 |
| 0.67 | hrs | 2295.5 | 25955 | 290.5 |
| 1 | hr | 346 | 11680 | 163.6 |
| 1.25 | hrs | 972.1 | 15065 | 182.6 |
| 1.5 | hrs | 465.3 | 12225 | 150.6 |
| 2 | hrs | 1876.3 | 14675 | 141.9 |
| 3 | hrs | 336.5 | 10745 | 105.3 |
| 4 | hrs | 398.4 | 7500 | 79.5 |
| 6 | hrs | 1006.4 | 11626.7 | 120.5 |
| 12 | hrs | 250.1 | 8002.7 | 87.2 |
| 24 | hrs | 224.2 | 5073.3 | 39.9 |

| | | | |
|---|---|---|---|
| Cₘₐₓ average ± | 8,690 ± 5,109 | 29,250 ± 6,914 | 324 ± 129 |
| standard deviation | | | |
| AUC average ± | 11,891 ± 3,053 | 189,972 ± 79,960 | 1,964 ± 895 |
| standard deviation | | | |
| (ng · hr/ml) | | | |
| 0 to 24 | | | |
| hours | | | |
| AUC average ± | 12,504 ± 3,712 | 270,813 ± 151,299 | 2,493 ± 1,214 |
| standard deviation | | | |
| (ng · hr/ml) 0 to | | | |
| infinite hours | | | |

Results

Tables 2, 3 and 4 show a summary of the PK parameters following IV and oral gavage dosing of Compound 1 at 2 and 75 mg/kg, respectively. Glu A and B were present in plasma, with Glu A being the predominant form having almost 66 times higher systemic exposure than Glu B following oral gavage dosing. Plasma levels of Compound 1 are 2% and 6% the level of the metabolites following IV and oral administration respectively, showing that Compound 1 undergoes extensive glucuronidation, irrespective of the route of administration.

After oral administration of Compound 1 at 75 mg/kg, the local absorption ratio (Fa) (i.e. fraction of Compound 1 absorbed in an intact form from the intestinal tract into the portal system) of Compound 1 was estimated using Equation (2) to be 48±18%.

The systemic oral bioavailability of Compound 1 following oral administration of Compound 1 at 75 mg/kg was estimated using Equation (2) to be 19±5%. The calculated hepatic recovery ratio (Fh) using Equation (3) was 40%. At the portal system, the mean ratio of Glu A to the Compound 1 was 20, while the ratio of Glu B to the Compound 1 was only 0.2. The magnitude of these values agreed with the ratios of metabolites in the jugular vein, where the ratio of Glu A to Compound 1 was 78 and the ratio of Glu B to Compound 1 was 1.

Given the extent of first pass metabolism of Compound 1 in both the gut (52%) and the liver (60%) and the clear evidence of enterohepatic recirculation, the results have demonstrated that the therapeutic effectiveness of a therapeutic agent can be boosted by such recirculation.

II. In Vitro Conversion of Glucuronide Conjugates by β-Glucuronidase

Glu A (500 μM) and Glu B (500 μM) were incubated with β-glucuronidase at 37° C. in pH 5.0 and pH 7.4 buffers. The pH 7.4 buffer is representative of the in vivo pH range in the GI tract. The results of each treatment are shown in Tables 5 (pH 5.0) and Table 6 (pH 7.4).

Glu A was prepared fresh in these experiments. A time-dependent incubation of Glu A and Glu B was carried out for 2, 4 and 6 hours.

TABLE 5
Conversion at pH 5.0

| Glucuronide | 0 hr | 0.5 hrs | 1 hr | 18 hrs |
|---|---|---|---|---|
| Glu-A | 100% Glu-A | 60% Glu-A | 34% Glu-A | 0% Glu-A |
| | 0% Cpd 1 | 40% Cpd 1 | 66% Cpd 1 | 100% Cpd 1 |
| Glu-B | 100% Glu-B | 100% Glu-B | 100% Glu-B | 100% Glu-B |
| | 0% Cpd 1 | 0% Cpd 1 | 0% Cpd 1 | 0% Cpd 1 |

TABLE 6
Conversion at pH 7.4

| Glucuronide | 0 hr | 2 hrs | 4 hrs | 6 hrs |
|---|---|---|---|---|
| Glu-A | 100% Glu-A | 67% Glu-A | 48% Glu-A | 23% Glu-A |
| | 0% parent | 33% parent | 52% parent | 77% parent |
| | drug | | | |
| Glu-B | 100% Glu-B | 100% Glu-B | 100% Glu-B | 100% Glu-B |
| | 0% parent | 0% parent | 0% parent | 0% parent |

Results

As shown in Table 5 and Table 6, Glu-A was hydrolyzed back to the Compound 1 following incubation with β-glucuronidase in a time-dependent manner at both pH 5.0 and pH 7.4. There was 100% conversion to Compound 1 by 18 hours of incubation at pH 5.0 and 77% conversion at 6 hours at pH 7.4. Comparatively, the conversion of Glu A to Compound 1 is less rapid at pH 7.4 than at pH 5.0.

In contrast to Glu A, Glu-B was not converted at either pH (100% Glu B remaining) after 6 or 18 hours of incubation. In the control samples without β-glucuronidase, both Glu-A and Glu-B were stable and Compound 1 was not observed.

III. Pharmacokinetics Following In Vivo Co-administration of β-Glucuronidase

The average plasma concentration versus time of Compound 1, Glu A and Glu B following intraduodenal dosing (10 mg/kg; N=6) with and without β-glucuronidase is shown in Table 7 and 8, respectively.

TABLE 7
Intraduodenal Average Plasma Concentration With Enzyme (ng/ml)

| Sample | | Cpd 1 | Cpd 1-GluA | Cpd 1-GluB |
|---|---|---|---|---|
| 0 | hrs | 0.0 | 0.0 | 0.0 |
| 0.25 | hrs | 129.0 ± 41.4 | 6,452.2 ± 1,732.4 | 508.4 ± 342.1 |
| 0.5 | hrs | 107.1 ± 26.6 | 4,820.5 ± 1,438.5 | 502.6 ± 362.8 |
| 1 | hr | 75.6 ± 35.0 | 3,864.2 ± 962.1 | 675.7 ± 585.8 |
| 2 | hrs | 21.2 ± 12.5 | 812.0 ± 425.9 | 274.6 ± 259.7 |
| 3 | hrs | 12.9 ± 4.3 | 461.5 ± 208.5 | 271.1 ± 180.8 |
| 4 | hrs | 9.5 ± 3.1 | 479.8 ± 106.8 | 194.3 ± 165.1 |
| 5 | hrs | 3.7 ± 6.3 | 420.4 ± 74.1 | 145.7 ± 84.4 |
| 6 | hrs | 1.7 ± 2.7 | 461.5 ± 113.4 | 119.0 ± 108.4 |

| | | | |
|---|---|---|---|
| Cₘₐₓ average ± | 129.4 ± 41.1 | 6,452.2 ± 1,732.4 | 754.6 ± 508.7 |
| standard deviation | | | |
| AUC average ± | 177.4 ± 55.3 | 8,723.2 ± 2,132.6 | 1,767.6 ± 1,282.3 |
| standard deviation | | | |
| (ng · hr/ml) 0 to 6 | | | |
| hours | | | |

TABLE 8
Intraduodenal Average Plasma Concentration Without Enzyme (ng/ml)

| Sample | | Cpd 1 | Cpd 1-GluA | Cpd 1-GluB |
|---|---|---|---|---|
| 0 | hrs | 0.0 | 0.0 | 0.0 |
| 0.25 | hrs | 75.2 ± 30.3 | 665.7 ± 197.9 | 70.2 ± 29.3 |
| 0.5 | hrs | 55.1 ± 15.5 | 687.0 ± 178.2 | 72.4 ± 28.2 |
| 1 | hr | 18.7 ± 5.9 | 463.0 ± 163.9 | 51.2 ± 24.9 |
| 2 | hrs | 3.2 ± 0.6 | 118.5 ± 36.3 | 13.5 ± 5.5 |
| 3 | hrs | 1.5 ± 0.9 | 66.6 ± 42.0 | 7.1 ± 5.0 |
| 4 | hrs | 1.6 ± 0.7 | 88.2 ± 53.9 | 9.6 ± 6.7 |
| 5 | hrs | 0.0 | 83.1 ± 23.2 | 9.2 ± 4.1 |
| 6 | hrs | 0.0 | 90.2 ± 28.1 | 10.4 ± 4.0 |

| | | | |
|---|---|---|---|
| Cₘₐₓ average ± | 76.0 ± 29.0 | 717.3 ± 171.1 | 75.7 ± 27.4 |
| standard deviation | | | |
| AUC average ± | 60.3 ± 17.8 | 1,172.7 ± 361.2 | 127.7 ± 55.3 |
| standard deviation | | | |
| (ng · hr/ml) 0 to 6 | | | |
| hours | | | |

The plasma concentration versus time of Compound 1, Glu A and Glu B following a single intracolonic dosing (10 mg/kg; N=6) with and without β-glucuronidase is shown in Table 9 and 10, respectively.

TABLE 9
Intracolonic Average Plasma Concentration With Enzyme (ng/ml)

| Sample | | Cpd 1 | Cpd 1-GluA | Cpd 1-GluB |
|---|---|---|---|---|
| 0 | hrs | 0.0 | 0.0 | 0.0 |
| 0.25 | hrs | 129.0 ± 41.4 | 6,452.2 ± 1,732.4 | 508.4 ± 342.1 |
| 0.5 | hrs | 107.1 ± 26.6 | 4,820.5 ± 1,438.5 | 502.6 ± 362.8 |
| 1 | hr | 75.6 ± 35.0 | 3,864.2 ± 962.1 | 675.7 ± 585.8 |
| 2 | hrs | 21.2 ± 12.5 | 812.0 ± 425.9 | 274.6 ± 259.7 |
| 3 | hrs | 12.9 ± 4.3 | 461.5 ± 208.5 | 271.1 ± 180.8 |
| 4 | hrs | 9.5 ± 3.1 | 479.8 ± 106.8 | 194.3 ± 165.1 |
| 5 | hrs | 3.7 ± 6.3 | 420.4 ± 74.1 | 145.7 ± 84.4 |
| 6 | hrs | 1.7 ± 2.7 | 461.5 ± 113.4 | 119.0 ± 108.4 |

| | | | |
|---|---|---|---|
| Cₘₐₓ average ± | 4.0 ± 0.6 | 754.6 ± 508.7 | 79.4 ± 47.3 |
| standard deviation | | | |
| AUC average ± | 9.5 ± 2.5 | 1,767.6 ± 1,282.3 | 175.9 ± 123.1 |
| standard deviation | | | |
| (ng · hr/ml) 0 to 6 | | | |
| hours | | | |

TABLE 10
Intracolonic Average Plasma Concentration Without Enzyme (ng/ml)

| Sample | | Cpd 1 | Cpd 1-GluA | Cpd 1-GluB |
|---|---|---|---|---|
| 0 | hrs | 0.0 | 0.0 | 0.0 |
| 0.25 | hrs | 6.4 ± 11.5 | 665.7 ± 197.9 | 70.2 ± 29.3 |
| 0.5 | hrs | 8.0 ± 15.7 | 6,452.2 ± 1,732.4 | 72.4 ± 28.2 |
| 1 | hr | 8.7 ± 18.2 | 4,820.5 ± 1,438.5 | 51.2 ± 24.9 |
| 2 | hrs | 2.0 ± 3.1 | 3,864.2 ± 962.1 | 13.5 ± 5.5 |
| 3 | hrs | 1.1 ± 2.7 | 66.6 ± 42.0 | 7.1 ± 5.0 |
| 4 | hrs | 0.0 | 88.2 ± 53.9 | 9.6 ± 6.7 |
| 5 | hrs | 0.0 | 83.1 ± 23.2 | 9.2 ± 4.1 |
| 6 | hrs | 0.0 | 90.2 ± 28.1 | 10.4 ± 4.0 |

| | | | |
|---|---|---|---|
| Cₘₐₓ average ± | 9.5 ± 17.9 | 1552.5 ± 1046.0 | 140.2 ± 56.9 |
| standard deviation | | | |
| AUC average ± | 15.5 ± 24.9 | 5061.9 ± 1,656.0 | 498.7 ± 137.2 |
| standard deviation | | | |
| (ng · hr/ml) 0 to 6 | | | |
| hours | | | |

Duodenal Administration Results:

Duodenal administration of β-glucuronidase demonstrated a statistically significant increase (“boosting”) in the levels of Compound 1, Glu A and Glu B.

At a dose of 10 mg/kg, there was a 2.9 fold increase in the Compound 1 AUC from 60±18 ng.hr/ml (as shown in Table 8) with no enzyme present to 177±55 with enzyme present (p≦0.005) (as shown in Table 7). The Cₘₐₓ of Compound 1 increased from 75±30 ng/ml (as shown in Table 8) with no enzyme present to 129±41 ng/ml with enzyme present (as shown in Table 7). The Tₘₐₓ of Compound 1 at 30 minutes remained unchanged.

The AUC of Glu A increased by more than a factor of 7 from 1216±371 ng.hr/ml with no enzyme present to 8940±2144 ng.hr/ml with enzyme present (p≦0.005).

Intracolonic Administration Results:

Intracolonic administration of Compound 1 with and without β-glucuronidase showed no difference in exposure of Compound 1 (p=0.57, AUC of parent after intracolonic administration was low<16 ng.hr/mL), but there was a decrease (p≦0.01) in the levels of Glu A from 5,062±1656 to 1,768±1282 ng.hr/mL and Glu B from 499±137 to 176±123 ng.hr/mL without and with β-glucuronidase, respectively.

Furthermore, the AUC of Glu A following intracolonic dosing with no enzyme was 4.3 times higher than that following intraduodenal administration with no enzyme present (p≦0.005), which is consistent with absorption of Compound 1 in both the duodenum and colon but more extensive gut metabolism of Compound 1 in the colon than duodenum.

These pharmacokinetic data support the hypothesis that the compound is subject to enterohepatic recirculation and when it is exposed to intraduodenally administered β-glucuronidase in the gut lumen, the glucuronide is cleaved back to parent compound and the parent compound is reabsorbed into the enterocyte and subject to “second” pass gut and hepatic metabolism.

It is to be understood that the preceding description of the invention and various examples thereof has emphasized certain aspects. Numerous other equivalents not specifically elaborated on or discussed may nevertheless fall within the spirit and scope of the present invention or the following claims and are intended to be included.

What is claimed is:

1. A method for modulating the pharmacokinetics and metabolism of a therapeutic agent comprising the step of administering the therapeutic agent in a combination with an enzyme, whereby the enzyme is involved in the transformation of a metabolite of the agent to a therapeutically available form of the agent.

2. The method of claim 1, wherein the enzyme transforms a metabolite of the agent back to the agent.

3. The method of claim 1 wherein the step of administering is via oral administration of the therapeutic agent in a combination with the enzyme.

4. The method of claim 1, wherein the therapeutic agent is selected from the group consisting of an anticholesterol agent, a cancer therapeutic agent, an analgesic agent, a hormonal agent, an anti-psychotic agent, a selective estrogen receptor modulator, a retinoid, mycophenolic acid, a fluoroquinolone, an anticonvulsant agent, an anti-HIV agent, a blood glucose lowering agent, an antidiabetic agent, an anti-Parkinson's disease therapeutic agent and an antismoking agent.

5. The method of claim 1, wherein the therapeutic agent is selected from the group consisting of ezetimibe, irrinotecan, tipifarnib, acetominophen, morphine, codeine, hydromorphone, testosterone, dihydrotestosterone, estradiol, 17-alpha-ethynylestradiol, estriol, risperidone, tamoxifen, raloxifine, ciprofloxacin, topiramate, carisbamate, valproic acid, GW273293, zidovudine, troglitazone, muraglitazar, muraglitzar, tolcapone, nicotine and (6,7-dimethoxy-2,4-dihydro-indeno [1,2-c]pyrazol-3-yl)-(3-fluoro-phenyl)-amine.

6. The method of claim 5, wherein the therapeutic agent is (6,7-dimethoxy-2,4-dihydro-indeno[1,2-c]pyrazol-3-yl)-(3-fluoro-phenyl)-amine.

7. The method of claim 1, wherein the enzyme is selected from N-oxide reductase, sulfoxide reductase, esterase, amidase, glucosidase, β-glucoronidase and sulfatase

8. The method of claim 7, wherein the enzyme is β-glucuronidase.

9. A product containing (a) a pharmaceutical composition containing a therapeutically effective amount of a therapeutic agent and (b) a pharmaceutical composition containing a constitutively effective amount of an enzyme as a combined preparation for simultaneous, separate or sequential use.

10. The product of claim 9, wherein the enzyme is selected from N-oxide reductase, sulfoxide reductase, esterase, amidase, glucosidase, β-glucoronidase and sulfatase

11. The product of claim 10, wherein the enzyme is β-glucuronidase.

12. A product containing (a) a pharmaceutical composition containing a therapeutically effective amount of a therapeutic agent that is subject to glucuronidation and (b) a pharmaceutical composition containing a constitutively effective amount of β-glucuronidase as a combined preparation for simultaneous, separate or sequential use.

13. A product containing (a) a pharmaceutical composition containing a therapeutically effective amount of a therapeutic agent that is subject to glucuronidation selected from the group consisting of an anticholesterol agent, a cancer therapeutic agent, an analgesic agent, a hormonal agent, an anti-depressent, an anti-psychotic agent, a selective estrogen receptor modulator, a retinoid, mycophenolic acid, a fluoroquinolone, an anticonvulsant agent, an anti-HIV agent, a blood glucose lowering agent, an antidiabetic agent, an anti-Parkinson's disease therapeutic agent and an antismoking agent and (b) a pharmaceutical composition containing a constitutively effective amount of an enzyme as a combined preparation for simultaneous, separate or sequential use.

14. The product of claim 13, wherein the enzyme is selected from N-oxide reductase, sulfoxide reductase, esterase, amidase, glucosidase, β-glucoronidase and sulfatase

15. The product of claim 14, wherein the enzyme is β-glucuronidase.

16. A product containing (a) a pharmaceutical composition containing a therapeutically effective amount of a therapeutic agent that is subject to glucuronidation selected from the group consisting of ezetimibe, irrinotecan, tipifarnib, acetominophen, morphine, codeine, hydromorphone, testosterone, dihydrotestosterone, estradiol, 17-alpha-ethynylestradiol, estriol, risperidone, tamoxifen, raloxifine, ciprofloxacin, topiramate, carisbamate, valproic acid, GW273293, zidovudine, troglitazone, muraglitazar, muraglitzar, tolcapone, nicotine and (6,7-dimethoxy-2,4-dihydro-indeno[1,2-c]pyrazol-3-yl)-(3-fluoro-phenyl)-amine and (b) a pharmaceutical composition containing a constitutively effective amount of β-glucuronidase as a combined preparation for simultaneous, separate or sequential use.

17. A product containing (a) a pharmaceutical composition containing a therapeutically effective amount of (6,7-dimethoxy-2,4-dihydro-indeno[1,2-c]pyrazol-3-yl)-(3-fluoro-phenyl)-amine and (b) a pharmaceutical composition containing a constitutively effective amount of β-glucuronidase as a combined preparation for simultaneous, separate or sequential use.
